(19) United States
(12) Patent Application Publication
(10) Pub. No.: US 2022/0233074 A1
(43) Pub. Date: Jul. 28, 2022

(54) DEVICE FOR AN OCULAR TONOMETER, AND ARRANGEMENT, METHOD AND USES THEREOF

(71) Applicant: Icare Finland Oy, Vantaa (FI)

(72) Inventor: Ari Kukkonen, Helsinki (FI)

(73) Assignee: Icare Finland Oy, Vantaa (FI)

(21) Appl. No.: 17/615,534

(22) PCT Filed: May 29, 2020
(86) PCT No.: PCT/EP2020/065017

§ 371 Date: Nov. 30, 2021

(30) Foreign Application Priority Data
May 31, 2019 (FI) 20195460

(51) Int. Cl. A61B 3/16

(52) U.S. Cl. CPC A61B 3/165

(57) ABSTRACT

A device (102) for an ocular tonometer comprising a dispenser (106) for dispensing and ejecting at least one liquid droplet (302) to the cornea (304) of an eye from a distance to the eye, wherein the dispenser (106) is arranged to eject the at least one droplet (302) such that the droplet (302) causes applanation to the cornea (304). Corresponding eye pressure measuring device, arrangement and method are also disclosed.

### FIELD OF THE INVENTION

Generally, the present invention relates to intraocular pressure measurement arrangements and arrangements for delivering fluid to an eye. Particularly, however not exclusively, the present invention pertains to a device, arrangement, method and uses for delivering fluid to an eye and/or for conducting intraocular pressure measurements with ejection of fluid.

### BACKGROUND

Fluid tonometers have been presented in prior wherein they pertain to air-operable intraocular pressure (TOP) measurement arrangements. Arguably, the most common type of solution of the kind is based on blowing pressurized air continuously from an outlet, which pressurized flow of air is used to induce applanation to the cornea. Consequently, measurement of this applanation of the cornea may be used to deduce characteristics, such as IOP or corneal thickness via optical measuring means for example.

Coincidentally, the most studied and reliable tonometers are of the rebound-type, which are comparable and calibratable in relation to a Goldmann Applanation Tonometer. This is an important notion when considering operability and verifiability of the measurement of various other TOP measurement arrangements.

Intrinsically, patient comfort of the TOP measurement is an important factor. Usually the shorter and less irritable the activity for manipulating the cornea the less the user will notice and react to the measurement. Tonometers that induce continuous applanation are for this reason usually less efficient and less practical.

A further common characteristic of fluid tonometers is that they are rarely suitable to be implemented as handheld standalone devices. This is most of all due to the insurmountable fact that pressurizing air requires compression and pneumatic means of the like that take up considerable space.

Prior art solutions have been represented in prior patent applications. For example, U.S. patent application publication no. 2008242966 depicts an air pulse discharging device for a noncontact type tonometer, which is operated by a cylinder-piston arrangement for continuous discharge of air towards the eye. A rebound-type tonometer using probes is presented in the applicant's own patent U.S. patent application publication no. 2009306493. A non-invasive, continuous applanation tonometer based on the use of flexible contact lens with an inflatable applanating chamber is presented in U.S. patent application publication no. 4628938. A manually operated probe-based tonometer is disclosed in U.S. patent application publication no. 2003097052. A membrane-based tonometer is presented in GB patent application publication no. 2308462. U.S. patent application publication no. 2010016704 depicts a method and system for monitor a condition of an eye utilizing intraocular pressure measurement datum and the time datum for determining dispensation of medication to the eye. An eye drop drug dispenser is disclosed in U.S. patent application publication no. 2014228783.

### SUMMARY OF THE INVENTION

The objective of the embodiments of the present invention is to at least alleviate one or more of the aforementioned drawbacks evident in the prior art arrangements. The objective is generally achieved with a device, arrangement and method in accordance with the present disclosure.

An advantage of the present invention is that it allows for generation of a discrete fluid dose that is comparable to a probe of a probe tonometer when colliding with the surface of the eye. In this way, a fluid tonometer with rebound tonometer characteristics is attained with the present invention.

One other advantage of the present invention is that it utilizes small discrete droplets for measurements, which provides for a more comfortable measurement event than with fluid tonometers that are based on continuous flow of fluid to the cornea of the eye. Even if there is no actual pain the continuous fluid flow will be of discomfort to the patient, which will often lead to blinking and movement of the eye, which should be avoided during measurement.

In accordance with one aspect of the present invention a device for an ocular tonometer characterized, in that the device comprises
dispenser for dispensing and ejecting at least one liquid droplet to the cornea of an eye from a distance to the eye,
wherein the dispenser is arranged to eject the at least one droplet such that the droplet causes applanation to the cornea.

In accordance with one aspect of the present invention an eye pressure measuring device utilizing the device of claim 1.

In accordance with one aspect of the present invention an arrangement for measuring intraocular pressure of an eye characterized, in that the arrangement comprises
a device for an ocular tonometer comprising
   dispenser for dispensing and ejecting at least one liquid droplet to the cornea of an eye from a distance to the eye, wherein the dispenser is arranged to eject the at least one droplet such that the droplet causes applanation to the cornea,
optical arrangement for measuring a deformation characteristic from the applanation of the cornea.

In accordance with one aspect of the present invention a method for exciting the cornea for intraocular pressure measurement characterized, in that the method comprises
dispensing and ejecting at least one liquid droplet to the cornea of an eye from a distance to the eye such that the droplet causes applanation to the cornea.

In accordance with another aspect of the present invention use of the device for delivering a droplet of liquid drug or a droplet comprising an amount of liquid drug to an eye.

As briefly reviewed hereinbefore, the utility of the different aspects of the present invention arises from a plurality of issues depending on each particular embodiment.

Different embodiments of the present invention are also disclosed in the attached dependent claims.

The expression “a number of” may herein refer to any positive integer starting from one (1). The expression “a plurality of” may refer to any positive integer starting from two (2), respectively.

The term “exemplary” refers herein to an example or example-like feature, not the sole or only preferable option.

The expressions “ejection” and “eject” are used to refer to the act of driving out, forcing out or expelling, as from a place or position, an amount of fluid.

The expressions “excitation” and “to excite” are used to refer to the action of applying force to the cornea, which in turn causes applanation and counterforce at and of the cornea.

### BRIEF DESCRIPTION OF THE DRAWINGS

Some exemplary embodiments of the present invention are reviewed more closely with reference to the attached drawings, wherein

FIG. 1 depicts a general view of the operating concept of the arrangement in accordance with the present invention,

FIGS. 2a, 2b and 2c depict different embodiments of the device in accordance with the present invention,

FIGS. 3a and 3b depict details of different embodiments of the dispenser in accordance with the present invention,

FIGS. 4a and 4b depict general views of a tonometer device utilizing an arrangement in accordance with the present invention,

FIGS. 5a, 5b, 5c and 5d depict the operating principle of the arrangement in accordance with the present invention,

FIG. 6 is a flowchart illustrating an embodiment of the method in accordance with the present invention,

FIG. 7 illustrates a setup for testing the dispenser in accordance with the present invention,

FIGS. 8a and 8b depict graphs illustrating measurement of deviation in accordance with different measurements with the setup of FIG. 7,

FIG. 9 illustrates a setup for IOP measurement with a rebound tonometer,

FIGS. 10a and 10b depict graphs illustrating measurement of deviation in accordance with different measurements with the setup of FIG. 9.

### DETAILED DESCRIPTION OF THE EMBODIMENTS

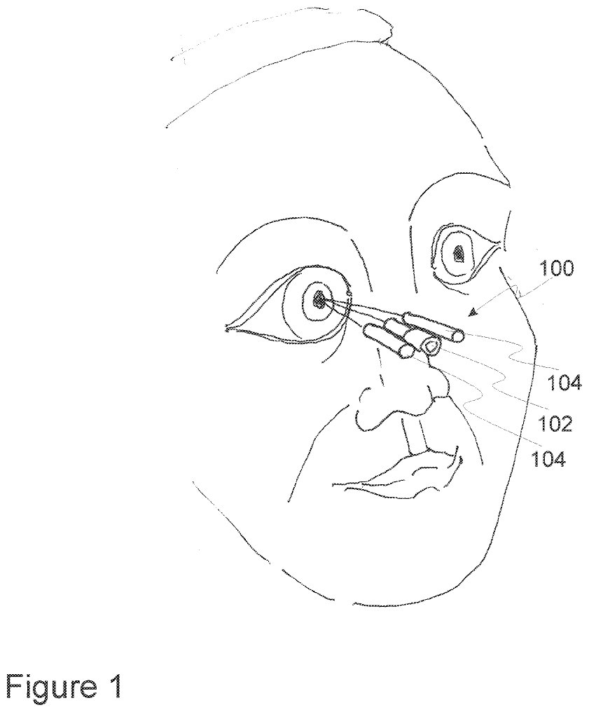

FIG. 1 depicts a general view of the operating concept of an arrangement (100) in accordance with the present invention. The arrangement (100) comprises a device (102) comprising a dispenser for dispensing and ejecting at least one liquid droplet to the cornea of an eye from a distance to the eye. The dispenser of the device (102) is arranged to eject the at least one droplet such that the droplet excitates and causes applanation to the cornea. The arrangement (100) further comprises, in addition to the device (102), an optical arrangement (104), which may comprise e.g. an emitter and a receiver, for measuring a deformation characteristic of the excitated cornea.

The optical arrangement (104) may additionally comprise an arrangement for producing an alignment pattern for aiming the dispensing of the droplet and the IOP measurement to the cornea.

The device (102) of the arrangement (100) is placed at a distance from the eye, which distance is preferably in the range of 4-10 mm.

The arrangement (100) is preferably arranged to operate in less than 50 ms the complete operation comprising delivering at least one droplet to an eye and measuring a deformation characteristic of the excitated cornea of the eye.

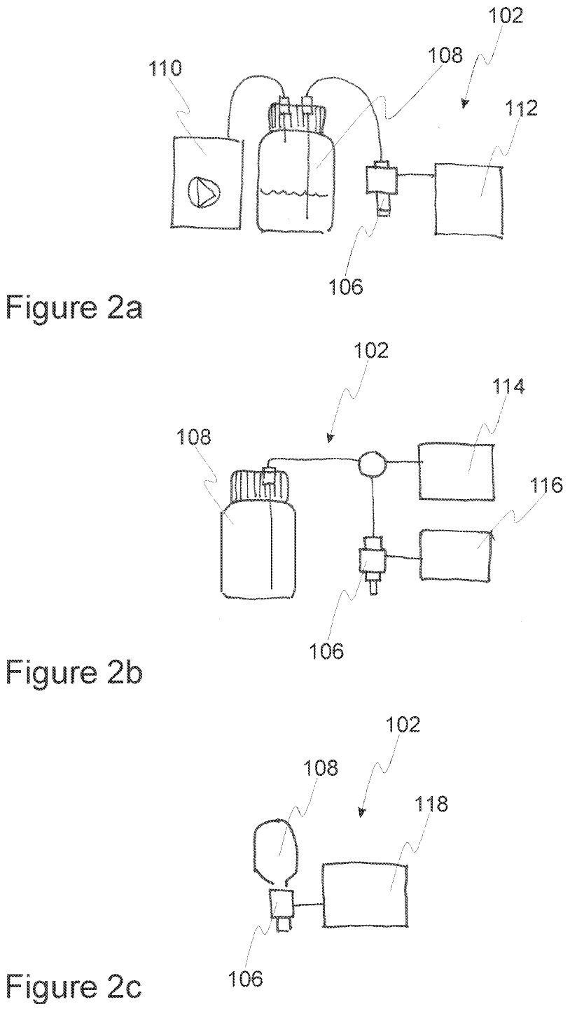

FIGS. 2a, 2b, 2c depict different embodiments of the device (102) in accordance with the present invention. The device (102) preferably comprises or is connected to at least a reservoir (108), means to produce force or pressure and/or control the dispensing of the liquid droplets from the dispenser (106).

FIG. 2a depicts a dispenser (106) connected to a reservoir (108), and means to produce and/or control pressure, wherein the reservoir (108) is connected to a pressure controller (110). The pressure controller (110) is used to raise the pressure in the reservoir (108) to e.g. approximately 0.4 bar. The dispenser (106) comprises a valve control solenoid, which is controlled with a solenoid controller (112), for dispensation and ejection of the liquid pressurized at the reservoir (108). The solenoid may be arranged to be opened for approximately 5 milliseconds at a time to produce the dispensation and ejection of the pressurized liquid with a predetermined or preferred velocity, measure of liquid, kinetic energy or momentum.

FIG. 2b depicts the dispenser (106) connected to a reservoir (108). In this embodiment a regulator (114) or pump is used to control the amount of liquid inserted into the dispenser (106) and to the conduit leading to the dispenser (106) from the reservoir. The device (102) may also comprise a system for bleeding air out of the conduits and the dispenser (106). The dispenser (106) comprises a piezo actuator which is connected with a controller (116) for controlling the operation of the piezo for facilitating actuation to the dispenser (106) for dispensation and ejection of the liquid from the dispenser (106) with a predetermined or preferred velocity, measure of liquid, kinetic energy or momentum.

FIG. 2c depicts the dispenser (106) as an integrated cassette-type device (102) wherein the liquid is stored in a reservoir (108) comprised essentially in or as part of the dispenser (106). The dispenser (106) comprises a microchannel or a piezo for facilitating dispensation and ejection of the liquid from the dispenser (106) with a predetermined or preferred velocity, measure of liquid, kinetic energy or momentum, which are controlled with a controller (118) at least functionally connected with the dispenser (106). The cassette-type device (102) may be a single-shot or multiple-shot action device (102), such that one device (102) may be used for only one IOP measurement or for another preferred amount of TOP measurements.

The dispenser (106) according to any of the embodiments may be arranged to dispense and eject at least one droplet having the volume of 1-10 microliters or preferably the volume of 3-5 microliters. The liquid comprises a composition similar to tears, which liquid may additionally or alternatively comprise an amount of administrable drug to an eye. The liquid of the droplets is preferably transparent.

The device (102) may also comprise means for rinsing the dispenser (106).

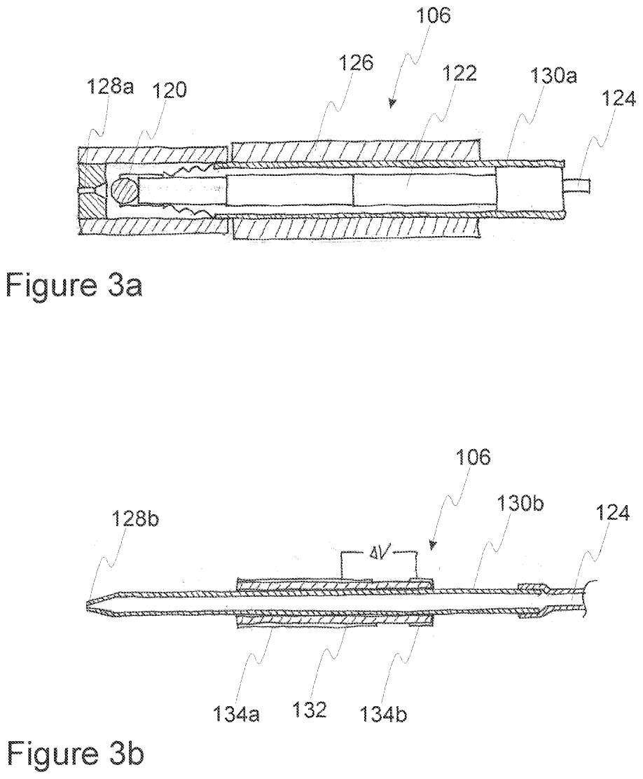

FIGS. 3a and 3b depict details of different embodiments of the dispenser (106) in accordance with the present invention.

FIG. 3a depicts a dispenser (106) design wherein droplets are dispensed and ejected by solenoid actuation. The dispenser (106) herein comprises a tubular shaped outer wall (130a) with a ruby ball-valve (120) operated by a solenoid. The dispenser (106) is fed with pressurized liquid by a conduit (124) connected to a reservoir. A coil (126) surrounding the outer wall (130a) is fed with current, wherefrom the movement of the solenoid plunger (122) is controlled, to let out an amount of pressurized liquid from the small orifice (128a). The orifice (128a) may be a sapphire orifice and comprise e.g. a diameter of 0.15-0.6 mm.

FIG. 3b depicts a dispenser (106) design wherein droplets are dispensed and ejected by piezo actuation. The dispenser (106) herein comprises a tubular shaped outer wall (130b) comprising glass. The dispenser (106) may be fed with pressurized liquid by a conduit (124) connected to a reservoir. Alternatively, the liquid may be directly stored as pressurized in the dispenser. Piezoelectric material (132) surrounding the glass tube is fed with current by electrodes (134a, 134b), which causes the piezoelectric material (132) to squeeze against the glass tube, which further causes a liquid droplet to squeeze out of the small orifice (128b) in the glass tube. The glass orifice (128b) has e.g. the diameter of 0.02-0.12 mm. The orifice (128b) in this embodiment is not closed because the liquid in the glass tube is kept in place by capillary effect when no additional force is exerted to the dispenser (106) outer wall (130b) by the piezoelectric arrangement.

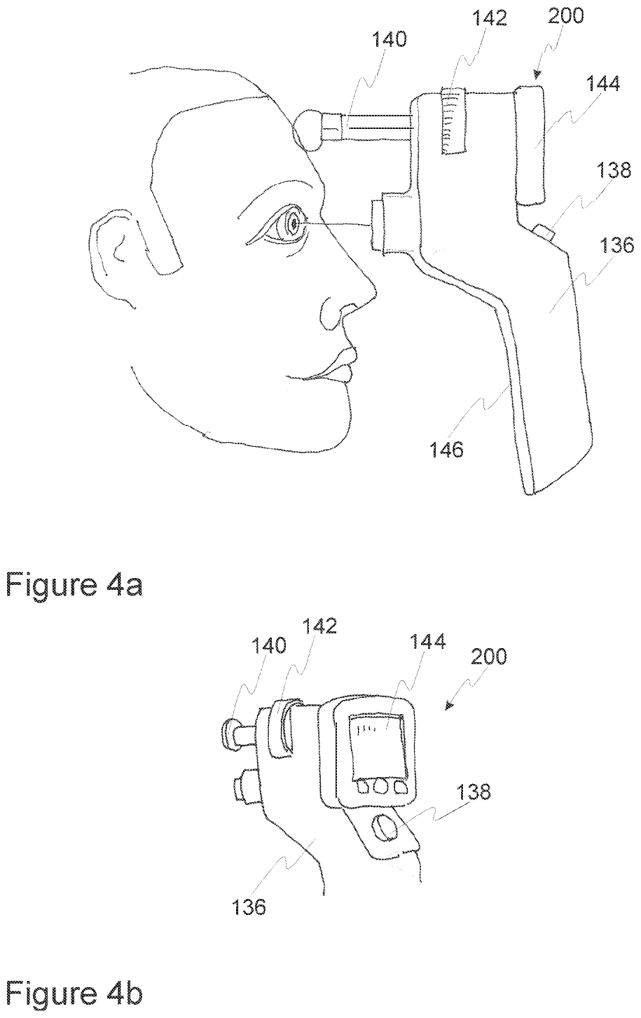

FIGS. 4a and 4b depict general views of a tonometer apparatus (200), i.e. an eye pressure measuring device, utilizing the arrangement (100) in accordance with the present invention. The FIGS. 4a and 4b depict one general application of the invention in a handheld tonometer apparatus (200).

The tonometer apparatus (200) is formed of a case component (136) made of a suitable material, inside of which all the components essential for the TOP measurement are fitted. The device (102) in accordance with present invention may be used in the apparatus (200) as a non-removable arrangement, which is fed with at least liquid, or as a single unit comprising a reservoir (108) and a dispenser (102) to dispense and eject at least one droplet at a pressure to a single direction. Such as a single unit may be a one-shot or a multiple-shot cartridge. The singe unit may be non-reusable or reusable. Hence the apparatus (200) may be arranged to facilitate insertion of such single units without need for a separate receptacle or external liquid conduit. The device (102) may comprise electrical or mechanical connection means for delivering mechanical force or current from the apparatus (200) the device (102) for facilitating dispensation and ejection of the droplet from the single unit, such as via pushing a button (138) or trigger of the apparatus (200).

In this embodiment, the case or body component (136) is essentially elongated and includes at its upper end a forehead support (140), which is used to adjust the distance from which the liquid droplet is ejected into the direction of the eye being measured. The forehead support (140) is specifically adjustable, e.g. by means of a wheel (142), which can be rotated manually.

The apparatus (200) further includes a display and control component (144), which is e.g. a liquid-crystal panel, in which the measurement result is displayed, and related control buttons etc. The apparatus (200) also comprises an operating switch (138), which when pressed, releases a liquid droplet towards the eye.

The operating power can be taken from dry cells or batteries, while the apparatus (200) additionally can have a socket, to which an external recharging device or power supply can be connected. A number of narrowings (146) may be used to make the apparatus (200) comfortable to use.

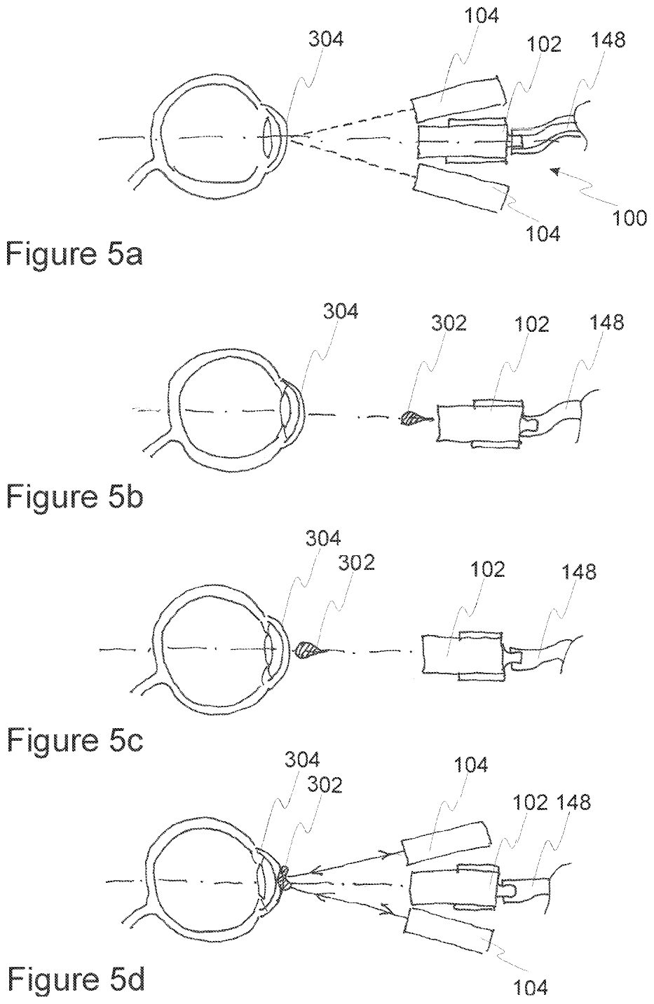

FIGS. 5a, 5b, 5c and 5d depict the operating principle of the arrangement (100) in accordance with the present invention. The figures depict ejection of a droplet (302) and the consequent applanation induced at the cornea (304) and deformation characteristic measurement thereof.

FIG. 5a depicts the arrangement being at a position and aligned with the cornea (304).

FIG. 5b depicts a liquid droplet (302) being ejected from the dispenser (102). The dispenser may be connected with control electronics and/or a liquid conduit (148) to control the dispensation and ejection of the liquid from the dispenser (102). The droplet is ejected at a measure of energy of ½ mv\hat{ }2.

Hence, different configurations in relation to the volume and type of liquid and velocity of the liquid can be used for setting a preferred energy for the ejected liquid droplet, which may be beneficial in view of different applications and distance between the dispenser (102) and the cornea (304). The volume of the droplet (302) may be chosen in view of the liquid properties, such as viscosity and/or surface tension and/or density. The kinetic energy or such measure of energy or momentum produced to the droplet is preferably similar to a probe of a rebound tonometer, such that the impact of the droplet to a surface of the eye causes similar deformation or is similar in its time of applanation to that of an rebound tonometer probe contacting the eye for measurement. Hence, measure of liquid and its ejection velocity may be optimized in view of the preferred amount of excitation aimed to produce to an eye, which optimization may be done in view of rebound tonometry, which has been widely discussed in the art of tonometry.

FIG. 5c depicts the droplet (302) traveling to the cornea (304) of an eye before collision.

FIG. 5d depicts the droplet (302) excitating the cornea (304) of an eye. After collision between the droplet (302) and the cornea (304) the cornea (304) is curved in accordance with the energy of the droplet (302), the corneal stiffness and TOP of the eye. The optical arrangement (104) is arranged to detect a measure of deformation induced by the droplet (302) to the cornea (304). The optical arrangement (104) may preferably be based on measuring reflection at the cornea (304), (change of) distance to the cornea (304) or Optical Coherence Tomography (OCT).

In some embodiments the optical arrangement (104) may also comprise or be based on imaging of the eye. In this type of optical arrangement a set of images of the cornea (304) are captured from which a deformation and topology thereof are detected, optionally together with the information of the imaging arrangement settings at the time of the images.

In accordance with another embodiment a grid may be produced on the eye e.g. by scanning, which grid may be used to provide topographical information of the deformation characteristic of the cornea (304).

The magnitude of applanation as well as the amount and speed of recovery of the cornea (304) may be used to determine properties of the cornea (304) and the deformation characteristic.

The optical arrangement (104) is preferably arranged to operate at a nonvisible spectrum. However, the optical arrangement (104) may further comprise a visible alignment pattern, such as a crosshair, for aligning the dispenser (102) ejection with the cornea (304) and to target the measurement thereof. The optical arrangement (104) may comprise also a fixation target for the patient, optionally to allow the patient to align the dispenser ejection with the cornea (304) of the patient. A feasible fixation target is presented e.g. in the PCT publication no. WO 2014/202840 A1.

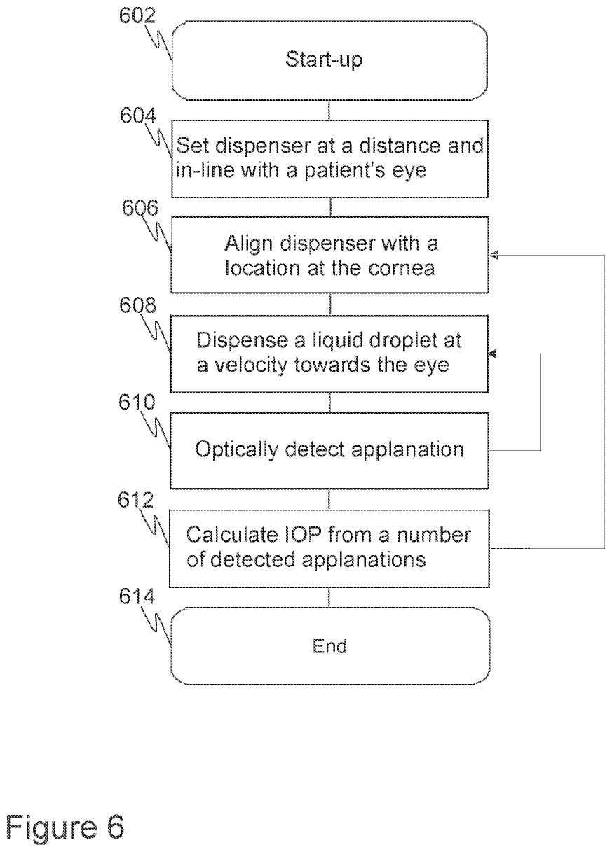

FIG. 6 is a flowchart illustrating an embodiment of the method in accordance with the present invention.

At 602, referred to as the startup the arrangement functions may be set, checked and/or calibrated.

At 604, the dispenser is set at an operating distance and in-line with a patient's eye. The operator of the device may be a physician carrying out a measurement to a patient or in some embodiments the patient may conduct the measurement to themselves.

At 606, the dispenser is aligned with a location of the cornea wherefrom the TOP measurement is to be made.

At 608, a liquid droplet of mass m is dispensed and ejected at velocity v towards the location of the cornea. The dispensation may be controlled to produce a liquid droplet of a preferred size or volume, such as 1-10 microliters or 3-5 microliters, and mass depending on the volume and used liquid, such as saline water having a similar composition to tears or other preferred dispensed liquid. The dispensation and ejection may be made simultaneously, such that the ejection is produced as a pulse which also dictates the amount (volume) that is dispensed.

At 610, the applanation caused by the liquid droplet is detected. The detected deformation characteristic of the applanation may encompass one or more different characteristics of cornea deformation, e.g. the measurement may comprise the amount or shape of deformation or the speed of deformation of the eye or cornea. The detection of applanation is done optically without physical contact with the cornea.

At 612, the TOP of the eye may be calculated from one or more of detected applanations. A number of applanations and deformation measurements may be made for calculating TOP of the eye. The dispenser may be also realigned in case the measurements are found inaccurate caused by measurement location.

At 614, the measurements and calculated TOP results may be saved, displayed to the operator or transmitted to another device.

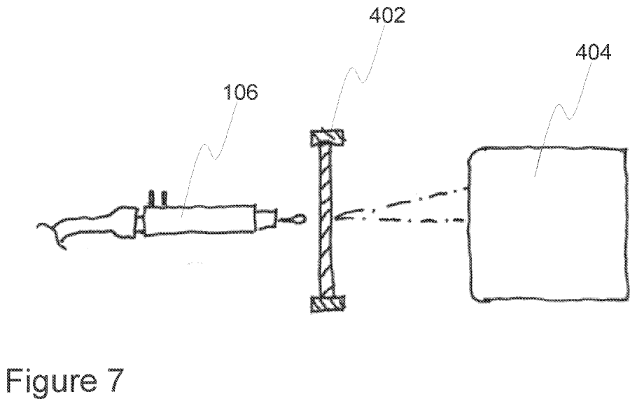

FIG. 7 illustrates a setup for testing the dispenser (106) in accordance with the present invention. The setup comprises a solenoid-operated dispenser (106), which is aimed to be fired at an artificial eye (402). The artificial eye (402) in this setup comprises a silicone membrane with tension. The deformation at the artificial eye (402) caused by the droplet dispensed and ejected from the dispenser (106) is detected and measured by an optical arrangement (404), which in this setup comprises a CCD Laser displacement sensor Keyence LK-G32.

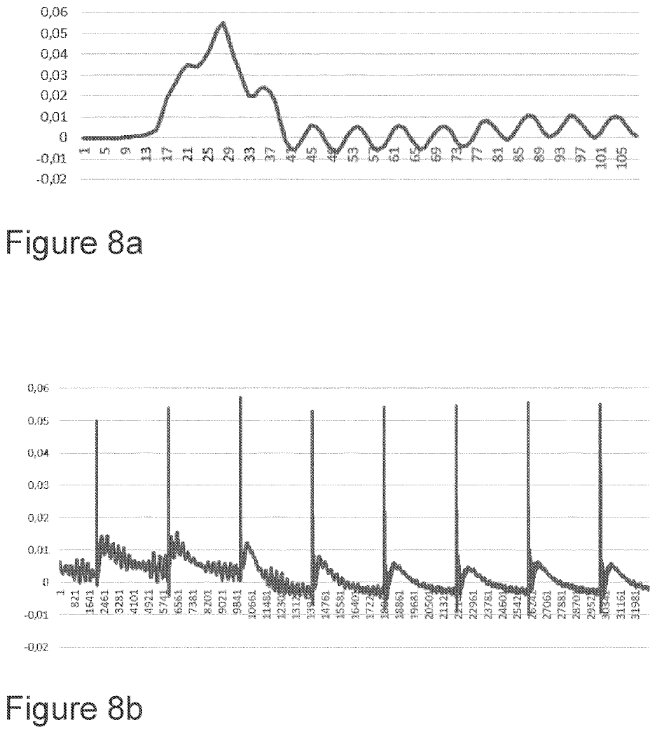

FIGS. 8a and 8b depict graphs illustrating measurement deviation in accordance with different measurements with the setup of FIG. 7.

FIG. 8a depicts deviation of the artificial eye (402) with a 4 μl droplet ejected at 17 mmHg pressure y=mm, x=samples @ 0.25 ms.

FIG. 8b depicts deviation of the artificial eye (402) with several 4 μl droplets ejected at 17 mmHg pressure y=mm, x=samples @ 0.25 ms.

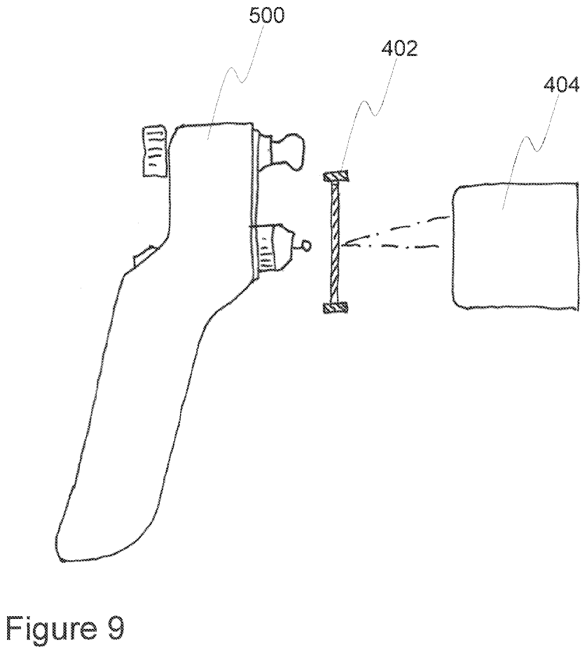

FIG. 9 illustrates a setup for TOP measurement with a rebound tonometer (500) for comparison of measurements results with the dispenser (106) in accordance with the present invention. The setup comprises a rebound tonometer Icare tonometer ic100, which is aimed to be fired at an artificial eye (402). The artificial eye (402) in this setup comprises a silicone membrane with tension. The deformation at the artificial eye (402) caused by the probe from the rebound tonometer (500) is detected and measured by the optical arrangement (404), which in this setup comprises a CCD Laser displacement sensor Keyence LK-G32.

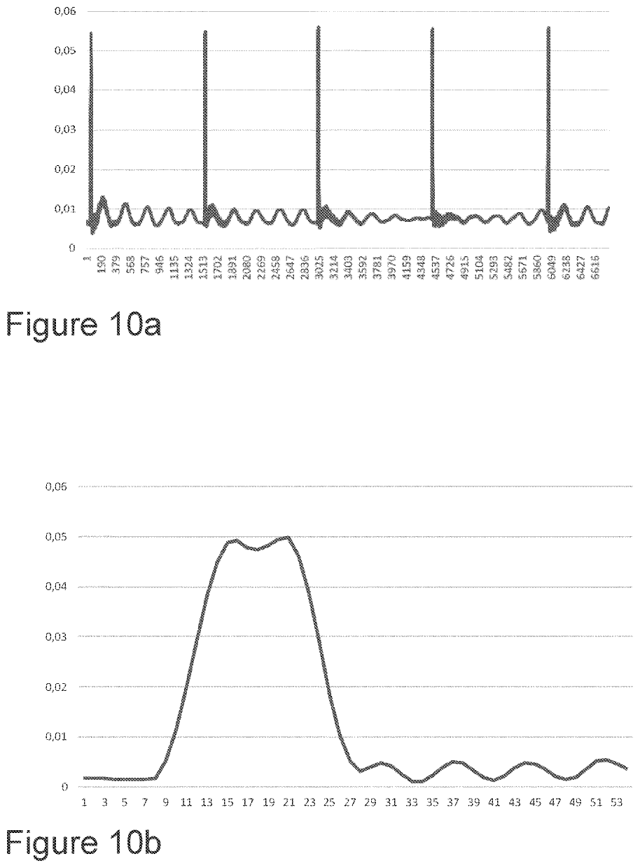

FIGS. 10a and 10b depict graphs illustrating measurement deviation in accordance with different measurements with the setup of FIG. 9.

FIG. 10a depicts deviation of the artificial eye (402) with a measurement of the rebound tonometer (500) at 17 mmHg pressure y=mm, x=samples @ 0.25 ms.

FIG. 10b depicts of the artificial eye (402) with several measurements of the rebound tonometer (500) at 17 mmHg pressure y=mm, x=samples @ 0.25 ms.

The scope of the invention is determined by the attached claims together with the equivalents thereof. The skilled persons will again appreciate the fact that the disclosed embodiments were constructed for illustrative purposes only, and the innovative fulcrum reviewed herein will cover further embodiments, embodiment combinations, variations and equivalents that better suit each particular use case of the invention.

1-19. (canceled)

20. A device for an ocular tonometer characterized, in that the device comprises
dispenser for dispensing and ejecting at least one liquid droplet to the cornea of an eye from a distance to the eye,
wherein the dispenser is arranged to eject the at least one droplet such that the droplet causes applanation to the cornea.

21. The device of claim 20, wherein the dispenser comprises or is connected to a liquid reservoir.

22. The device of claim 21, wherein pressure for the ejection of the droplet is caused by a pressure induced in the reservoir.

23. The device of claim 20, wherein the dispenser comprises solenoid valve control for droplet dispensing and ejecting.

24. The device of claim 20, wherein the dispenser comprises piezo control for droplet dispensing and ejecting.

25. The device of claim 20, wherein the dispenser comprises a microchannel and pressure control thereof for droplet dispensing and ejecting.

26. The device of claim 20, wherein the dispenser is arranged to dispense and eject at least one droplet having the volume of 1-10 microliters.

27. The device of claim 20, wherein the dispenser is arranged to dispense and eject at least one droplet having the volume of 3-5 microliters.

28. An eye pressure measuring device utilizing the device of claim 20.

29. An arrangement for measuring intraocular pressure of an eye characterized, in that the arrangement comprises
a dispenser for dispensing and ejecting at least one liquid droplet to the cornea of an eye from a distance to the eye, wherein the dispenser is arranged to eject the at least one droplet such that the droplet causes applanation to the cornea,
optical arrangement for measuring a deformation characteristic from the applanation of the cornea.

30. The arrangement of claim 29 wherein the optical arrangement comprises an optical distance measurement arrangement.

31. The arrangement of claim 29, wherein the optical arrangement comprises a light reflection measurement arrangement.

32. The arrangement of claim 29, wherein the optical arrangement comprises an Optical Coherence Tomography arrangement.

33. The arrangement of claim 29, comprising an optical arrangement for producing an alignment pattern.

34. The arrangement of claim 29, wherein the optical arrangement for measuring a deformation characteristic is additionally used for producing the alignment pattern.

35. The arrangement of claim 29, wherein the operation of delivering at least one droplet to an eye and measuring a deformation characteristic of the applanated cornea of the eye is executed in less than 50 milliseconds.

36. A method for exciting the cornea for intraocular pressure measurement characterized, in that the method comprises
dispensing and ejecting at least one liquid droplet to the cornea of an eye from a distance to the eye such that the droplet causes applanation to the cornea.

37. The method of claim 36, comprising optically measuring a deformation characteristic from the applanation of the cornea.

38. The method of claim 37, comprising determining the intraocular pressure from the deformation characteristic.
